(19) United States
(12) Patent Application Publication
(10) Pub. No.: US 2023/0320979 A1
(43) Pub. Date: Oct. 12, 2023

(54) OIL-IN-WATER EMULSIONS FOR INTRAVITREAL ADMINISTRATION

(71) Applicant: SANTEN SAS, EVRY-COURCOURONNES (FR)

(72) Inventors: Mathieu SCHMITT, Paris (FR); Philippe DAULL, Soisy-sur-Seine (FR); Jean-Sébastien GARRIGUE, Verrières (FR)

(73) Assignee: SANTEN SAS, EVRY-COURCOURONNES (FR)

(21) Appl. No.: 18/043,939

(22) PCT Filed: Sep. 15, 2021
(86) PCT No.: PCT/EP2021/075275

§ 371 Date: Mar. 3, 2023

(30) Foreign Application Priority Data
Sep. 16, 2020 (EP) 20306037.1

(51) Int. Cl. A61K 9/00; A61K 9/107; A61K 47/44; A61K 47/26; A61K 47/14; A61K 31/573

(52) U.S. Cl. CPC A61K 9/0048; A61K 9/1075; A61K 47/44; A61K 47/26; A61K 47/14; A61K 31/573

(57) ABSTRACT

An oil-in-water emulsion for intravitreal administration, especially injection, including an active ingredient, an oil, a mixture of two non-ionic surfactants and water, the emulsion having a droplet size ranging from about 100 to about 200 nm. The emulsion avoids troubles of vision after administration into the vitreous body. The emulsion provides good therapeutic activity of the active ingredient for treating eye diseases or conditions, such as an eye disease or condition of the posterior segment of the eye.

### FIELD OF INVENTION

The present invention pertains to the field of ophthalmic compositions and relates to oil-in-water emulsions for intravitreal administration. In particular, the invention relates to translucent emulsions for intravitreal injection.

### BACKGROUND OF INVENTION

Delivering therapeutic agents to the posterior segment of the eye is a challenge. Especially, convenient treatments of diseases affecting the retina are not available due to the difficulty of efficiently administering an active ingredient, e.g., a corticosteroid drug, to the macula. Topical administration such as instillation (eye drops and the like) is mostly ineffective because of the limited ocular absorption through the cornea and sclera, and because of the removal of the active ingredient by tears and blinking. Methods involving direct administration into the vitreous body and in particular intravitreal injection have been considered, but they also present significant limitations. For example, a specific tissue of the posterior segment is difficult to target, or the compatibility of the ophthalmic vehicle with the vitreous body does not ensure safety of the treatment. Moreover, due to the short half-life of some active ingredients in the vitreous body, repeated injections are often necessary, which is inconvenient for the patient. Intravitreal administration of poorly water-soluble or hydrolysable active ingredients, such as long chain esters of prodrugs of corticosteroids, raises further difficulties: where hydrophilic agents may be injected in aqueous-based vehicles that are very similar to the vitreous gel, lipophilic agents generally cannot be solubilized in water. It is thus required to introduce in the vitreous body an oil component that is not naturally compatible therewith.

The Applicant developed injectable emulsions comprising lipophilic corticosteroid prodrugs disclosed in WO 2007/138113 A1 for the treatment of ophthalmic diseases affecting the posterior segment of the eye. Although these emulsions represented at this time a significant progress from existing treatment options, in particular in terms of reduction of ocular side effects (such as increase of intraocular pressure) and comfort of the patient (reduction of the number of injections), they did not fully address an important issue of intravitreal administration, namely the troubles of the vision. Indeed, the oil droplets that are the dispersed phase of an oil-in-water emulsion have a refractive index which differs substantially from that of the vitreous gel, so that they may form a haze between the lens and the retina following the injection of the emulsion into the vitreous body. Depending on the composition and size of the oil droplets, this may lead to various troubles of the vision such as blurring or other visual disturbances. Troubles of vision are unpleasant for the patient and may limit patient compliance, which is already a significant issue for intraocularly injected treatments. This problem is especially critical when the active ingredient is lipophilic because, in order to be able to solubilize the active ingredient into the oil, the emulsion has to comprise relatively high amounts of discontinuous phase (oil). High amount of oil in the emulsion before injection leads to high volume occupied by the droplets in the vitreous body. In other cases, the active ingredient may not be completely solubilized in the oil, so that the dispersed phase becomes blurred (“milky” aspect). As a consequence, troubles of the vision may occur due to the high volume of the droplets and/or the lack of translucency of the droplets.

Therefore, there is still a need for new ophthalmic vehicles for intravitreal administration, that would avoid the troubles of the vision and have good therapeutic efficacy, especially for the delivery of lipophilic compounds to the posterior segment of the eye. The Applicant carried out in-depth research regarding injectable ophthalmic vehicles and discovered that using an oil-in-water emulsion with a narrow range of droplet size had an effect both on limiting the blurring of vision and on the release of the active ingredient.

Although the droplet size could be adjusted by using mixtures of surfactants and oils known in the art, the Applicant realized that this was not sufficient to achieve proper control of the formation and behaviour of the droplets and the technical problems were not entirely solved by acting only on the droplet size range. The Applicant unexpectedly found out that the emulsion had to be specifically designed within a limited selection of components.

According to a first embodiment of the invention, the two non-ionic surfactants that are a mixture of (i) a polyoxyethylene castor oil and a sorbitan ester and/or (ii) a polyoxyethylene castor oil and a polysorbate; preferably a mixture of a polyoxyethylene castor oil and a sorbitan ester. According to a second embodiment of the invention, wherein the active ingredient is very lipophilic, a triglyceride oil is used as the dispersed phase of the emulsion.

The Applicant surprisingly found out these distinguishing features of the emulsions of the invention achieve significant increase of post-intravitreal administration transparency of the vitreous body, thereby leading to significant reduction of troubles or vision, or even the disappearance thereof. The emulsions of the invention also provide good therapeutic efficacy and allow controlled and sustained drug delivery from the vitreous to the back of the eye. Moreover, the emulsions of the invention can be sterilized and they are stable overtime.

### SUMMARY

This invention relates to an oil-in-water emulsion for intravitreal administration comprising: from about 0.01 to about 50% w/w an oil, from about 0.001 to about 10% w/w of an active ingredient comprised in said oil, from about 0.001 to about 25% w/w of a mixture of at least two non-ionic surfactants comprising (i) a polyoxyethylene castor oil and a sorbitan ester and/or (ii) a polyoxyethylene castor oil and a polysorbate, and water; wherein said emulsion has a droplet size ranging from about 100 to about 200 nm.

According to one embodiment, the oil-in-water emulsion has a light transmittance after being diluted in water in a ratio in volume emulsion/water of about 0.01 ranging from about 70% to about 100%, preferably ranging from about 75% to about 100%, more preferably ranging from about 80% to about 100%. According to one embodiment, the mixture of at least two non-ionic surfactants comprises a polyoxyethylene castor oil and a sorbitan ester. According to one embodiment, the mixture of at least two non-ionic surfactants comprises polyoxyl 35 castor oil and/or sorbitan monolaurate, preferably polyoxyl 35 castor oil and sorbitan monolaurate. According to one embodiment, the oil is selected from triglyceride oils; preferably from short chain triglycerides, medium chain triglycerides and long chain triglycerides; more preferably said oil is medium chain triglycerides. According to one embodiment, the active ingredient is a lipophilic active ingredient; preferably a long chain ester of a drug, more preferably a C₁₀-C₂₁ ester of a drug, furthermore preferably a C₁₂-C₁₆ ester of a drug, furthermore preferably a C₁₄ ester of a drug; preferably the drug is a steroid, more preferably a corticosteroid. In one embodiment, the active ingredient is selected from dexamethasone caprate, dexamethasone laurate, dexamethasone myristate, dexamethasone palmitate and dexamethasone stearate; preferably selected from dexamethasone laurate, dexamethasone myristate and dexamethasone palmitate; more preferably dexamethasone palmitate. According to one embodiment, the oil-in-water emulsion comprises medium chain triglycerides, an active ingredient selected from dexamethasone caprate, dexamethasone laurate, dexamethasone myristate, dexamethasone palmitate and dexamethasone stearate, polyoxyl 35 castor oil, sorbitan monolaurate, glycerol and water. In one embodiment, the active ingredient comprises dexamethasone palmitate. According to one embodiment, the oil-in-water emulsion is anionic. According to one embodiment, the oil-in-water emulsion has a droplet size ranging from about 110 to about 175 nm, preferably from about 120 nm to about 150 nm.

According to one embodiment, the oil-in-water emulsion is for use as a medicament. In one embodiment, the oil-in-water emulsion is for use in the treatment of an eye disease or condition; preferably a disease or condition of the posterior segment of the eye; more preferably a disease selected from uveitis, macular edema such as diabetic macular edema (DME), macular degeneration such as age related macular degeneration (AMD), retinal detachment, ocular tumors, bacterial infections, fungal infections, viral infections, multifocal choroiditis, diabetic retinopathy, proliferative vitreoretinopathy (PVR), sympathetic ophthalmia, Vogt Koyanagi Harada (VKH) syndrome, histoplasmosis, uveal diffusion and vascular occlusion. In one embodiment, the oil-in-water emulsion is intravitreally administered, preferably intravitreally injected, in an amount ranging from about 5 to about 250 μL, preferably ranging from about 10 to about 100 μL, more preferably ranging from about 25 to about 50 μL.

This invention also relates to an implantable device comprising the oil-in-water emulsion according to the invention. This invention also relates to a prefilled syringe comprising the oil-in-water emulsion according to the invention.

### Definitions

In the present invention, the following terms have the following meanings:
“About” is used herein to mean approximately, roughly, around, or in the region of. The term “about” preceding a figure means plus or less 10% of the value of said figure. When the term “about” is used in conjunction with a numerical range, it modifies that range by extending the boundaries above and below the numerical values set forth by 10%.
“Active ingredient” and “therapeutic agent” are synonyms and refer to a compound for therapeutic use, and relates to health. Especially, an active ingredient may be indicated for treating or preventing a disease or condition, as defined hereinafter. Preferably, the active ingredient is for use in the treatment of an eye disease or condition. In the present disclosure, active ingredients encompass both prodrugs and drugs.
“Alkyl” by itself or as part of another substituent refers to a hydrocarbyl radical of formula —CₙH₂ₙ₊₁ wherein n is a number greater than or equal to 1. Alkyl groups may be linear or branched. In this invention, alkyl groups typically comprise from 1 to 30 carbon atoms, preferably from 6 to 20 carbon atoms, more preferably from 8 to 18 carbon atoms, furthermore preferably from 10 to 16 carbon atoms.
“Alkenyl” as used herein refers to an unsaturated hydrocarbyl group, which may be linear or branched, comprising one or more carbon-carbon double bonds. Alkenyl groups may be linear or branched. In this invention, alkenyl groups typically comprise from 1 to 30 carbon atoms, preferably from 6 to 20 carbon atoms, more preferably from 8 to 18 carbon atoms, furthermore preferably from 10 to 16 carbon atoms. Alkenyl groups may for example comprise one or two carbon-carbon double bonds.
“Between [lower value] and [upper value]” and the like define a numerical range that excludes (i.e., does not encompass) both the upper value and the lower value.
“Corticosteroid” refers to any of a wide variety of drugs that are closely related to cortisol, a hormone which is naturally produced in the adrenal cortex. Non-limitative examples of corticosteroids include betamethasone, budesonide, cortisone, dexamethasone, hydrocortisone, methylprednisolone, prednisolone, prednisone and triamcinolone. In the invention, corticosteroids may be natural or artificial.
“Droplet size” of an emulsion refers to the peak droplet size or the mean droplet size, preferably the peak droplet size, of the dispersed phase. Droplet size may be measured by methods known in the art, e.g., by light scattering after dilution in water using a High-Performance Particle Sizer (such as a Zetasizer 2000 or a Zetasizer NanoS, Malvern Instruments, UK).
“Drug” or “pharmacologically active agent” refers to a substance that causes a therapeutic change in physiology or psychology of a subject, preferably a human, when administered. Preferably, the active ingredient is for treating an eye disease or condition. A drug may be administered to a subject per se, in the form of a pharmaceutically acceptable salt thereof, or in the form of a prodrug thereof.
“Emulsion”, in accordance with the general knowledge in the art, refers to a macroscopically homogeneous but microscopically heterogeneous mixture of two or more liquids that are normally immiscible owing to liquid-liquid phase separation. In an emulsion, one liquid (the “dispersed phase”) is dispersed in the other (the “continuous phase”) in the form of droplets. Stability of an emulsion is typically achieved by the inclusion of at least one surfactant in the emulsion. The surfactant may for example be present in the surface of the droplets of the emulsion.
“Hydrophilic lipophilic balance” or “HLB” refers to a parameter expressing the relative simultaneous attraction of a surfactant for dispersed and continuous phases (typically aqueous and oil phases) in an emulsion. Surfactants are characterized according to the balance between the hydrophilic and lipophilic portions of their molecules. HLB value indicates the polarity of the molecule in an arbitrary range from 1 to 40. Surfactants commonly used in emulsions typically have HLB values ranging from 1 to 20. The HLB increases with increasing hydrophilicity.
“Intravitreal administration” refers to the administration of a composition into the vitreous body (vitreous humor) of an eye. Intravitreal administration may for example be carried out by intravitreal injection.
“Intravitreal injection” refers to intravitreal administration by injection means, e.g., a syringe.
“Lipophilic” refers to a property of a compound that dissolves more readily in fats, oils, lipids and non-polar solvents than in water. Lipophilic character may for instance be quantified by means of log P values: a compound may for example be considered lipophilic when its log P is higher than 5, preferably higher than 6, more preferably higher than 7, furthermore preferably higher than 8.
“Log P” is defined as follows: The partition coefficient P of a compound is the ratio of the concentration of said compound (as a neutral molecule) in water to the concentration thereof in octanol. The logarithm of said ratio is called “log P”. Log P can be determined according to published procedures, e.g., measured with a suitable liquid chromatography (HPLC) method (Caron, J. C. and Shroot, B., Journal of Pharmaceutical Sciences, 1984, pp. 1703-1706) or calculated with a suitable in silico method such as XLogP3 method (Chen, T. et al., Journal of Chemical Information and Modeling, 2007, Vol. 47, No. 6, pp. 2140-2148).
“Long chain ester of a drug” refers to a chemical entity comprising an ester function (—COO—), wherein one among the carbon and oxygen atoms is covalently bound to an alkyl or alkenyl chain comprising at least 7 carbon atoms, and wherein the other among the carbon and oxygen atoms is covalently bound to or is part of a functional group of a drug. Long chain esters of drugs are useful as prodrugs. The alkyl or the alkenyl chain may be linear or branched and typically comprises 9, 10, 11, 12, 13, 14, 15, 16, 17, 18, 19 or 20 carbon atoms, preferably 9, 11, 13, 15 or 17 carbon atoms, more preferably 11, 13 or 15 carbon atoms. The alkyl or alkenyl chain may for example be an alkyl chain. A long chain ester of a drug thus comprises one more carbon atom than its alkyl or alkenyl chain (being the carbon of the ester functional group), so that for example a “C₁₂-C₁₆ ester” has an alkyl or alkenyl chain comprising from 11 to 15 carbon atoms. A long chain ester of a drug typically results of the reaction of esterification between an alcohol function of the drug and the carboxylic acid function of a fatty acid, according to synthetic methods or natural processes well-known in the art. Non-limitative examples of fatty acids are decanoic (capric) acid (10:0), dodecanoic (lauric) acid (12:0), tetradecanoic (myristic) acid (14:0), hexadecanoic (palmitic) acid (16:0) and octadecanoic (stearic) acid (18:0).
“Microemulsion” refers to an emulsion wherein the droplet size ranges from 1 to 100 nm, usually from 10 to 50 nm, in accordance to IUPAC's definition.
“Oil-in-water emulsion” refers to an emulsion wherein the dispersed phase is significantly more lipophilic than the continuous phase. Typically, the dispersed phase is oil-based and/or the continuous phase is water-based.
“Ophthalmic substance” or “ophthalmic composition” refers to a substance or a composition intended to be administered to the eye of a subject and/or which is suitable to be administered to the eye of a subject. Preferably, an ophthalmic substance or composition presents a pharmaceutical effect, i.e., is indicated for treating an eye disease or condition.
“Ophthalmic treatment” refers to a treatment of an eye disease or condition in a subject in need thereof, which comprise a step of administration of an ophthalmic substance or composition to the eye of a subject.
By “pharmaceutically acceptable” referring to a substance or a composition, it is meant that the substances or the composition are compatible with each other and/or not deleterious to the subject, preferably a human, to which the substance or composition is administered. Especially, it does not produce an adverse, allergic or other untoward reaction when administered to a subject, preferably a human. For human administration, compositions should meet sterility, pyrogenicity, general safety and purity standards as required by regulatory offices, such as, for example, Food and Drug Administration (FDA) Office or European Medicine Agency (EMA).
“Pharmaceutically acceptable excipient” refers to a pharmaceutically acceptable excipient, carrier or vehicle. It includes any and all solvents, dispersion media, coatings, antibacterial and antifungal agents, isotonic and absorption delaying agents, and the like.
“Pharmaceutical composition” refers to a composition comprising at least an active ingredient in association with at least a pharmaceutically acceptable excipient. A pharmaceutical composition is for therapeutic use, and relates to health. Especially, a pharmaceutical composition may be indicated for treating or preventing a disease or condition, as defined hereinafter. Preferably, a pharmaceutical composition is for use in the treatment of an eye disease or an eye condition.
“Posterior segment” or “posterior cavity” of the eye refers to the back two-thirds of the eye that include the anterior hyaloid membrane and all of the optical structures behind it, including the vitreous body, the retina, the retinal pigment epithelium (RPE), the choroid and the optic nerve.
“Prodrug” refers to an active ingredient that, after administration, is converted within the body (i.e., metabolized) into a drug (i.e., a pharmacologically active agent). Typical prodrugs are ester, ether and amide derivatives of drugs.
“Ranging from [lower value] to [upper value]” and others similar recitations define a numerical range that includes (i.e., encompasses) both the upper value and the lower value. Moreover, any range so defined in the present application should be construed as including an explicit disclosure of the corresponding narrower range “between [lower value] and [upper value]”.
“Steroid” refers to a hormone normally produced by the adrenal glands which are two small glands found above the kidneys. Steroids have four cycloalkyl fused rings arranged in a specific molecular configuration. In the invention, a steroid may be natural or artificial.
“Subject” refers to a warm-blooded animal, preferably a mammal, more preferably a human. Preferably, the subject is a patient, i.e., a subject who is awaiting the receipt of, or who is receiving medical care, or who is/will be the object of a medical procedure. Preferably, the subject has an eye disease or an eye condition.
“Translucency” or “translucence” or “translucidity” are synonyms and refer to the physical property of allowing light to pass through a material without appreciable scattering of light but wherein the photons do not necessarily follow Snell's law on the macroscopic scale: because the photons can be scattered at either of the two interfaces (e.g., air/material), or internally (within the material), where there is a change in index of refraction. By contrast, when the photons follow Snell's Law on a macroscopic scale, the translucent material is considered “transparent”. Typically, a translucent material is made up of components with different indices of refraction whereas a transparent material is made up of components with a uniform index of refraction. Translucency is the opposite of opacity. The invention concerns in particular the translucency or transparency of oil-in-water emulsions, of continuous or dispersed phases of emulsions and/or of the vitreous gel. Translucency or transparency may be determined by qualitative and/or quantitative methods known in the art, e.g., by means of observation in animal model, patient questionnaire or turbidity test.
“Treating” or “treatment” or “alleviation” refers to both therapeutic treatment and prophylactic or preventative measures; wherein the object is to prevent or slow down (lessen) the targeted disease or condition in a subject in need thereof. Those in need of treatment include those already with the disease or condition as well as those prone to have the disorder or those in whom the disorder is to be prevented. A subject is successfully “treated” for a disease or condition if, after receiving a therapeutic amount of a substance or composition, the subject shows observable and/or measurable effect on one or more of the following: reduction in the number of pathogenic cells; reduction in the percent of total cells that are pathogenic; relief to some extent, of one or more of the symptoms associated with the specific disease or condition; reduced morbidity and mortality; and/or improvement in quality of life issues. The above parameters for assessing successful treatment and improvement in the disease are readily measurable by routine procedures familiar to a physician. Preferably, the treatment involves a step of administration of an active ingredient, as defined hereinabove. In the invention, the disease or condition is an eye disease or an eye condition, i.e., a pathologic disorder or a condition affecting the eye of a subject.
“Turbidity” refers to the haziness or cloudiness of a fluid caused by large numbers of individual particles that are generally invisible to the naked human eye, similar to smoke in air. The invention concerns in particular the turbidity of oil-in-water emulsions and/or of the vitreous gel, wherein the individual particles are dispersed phase (oil) droplets. Turbidity may be determined by methods known in the art, e.g., by using a stability analyser (for example Turbiscan Beckman Coulter, US). Turbidity is expressed either in % light transmittance (up to 100% for completely translucent materials) or % light absorbance (up to 100% for completely opaque materials), wherein % light transmittance=100−% light absorbance.
“Zeta potential” is defined as follows: One well-known approach in the art to stabilize an emulsion is to confer an electrostatic charge to the droplets surface of the dispersed phase, which will result in more droplet repulsion and less droplet coalescence. Colloidal particles dispersed in an emulsion are electrically charged due to their ionic characteristics and/or dipole attributes. This charge is referred to in the art as the “zeta potential” and it reflects the magnitude of the repulsion or attraction between particles. Zeta potential can be measured by methods known in the art, e.g., by measuring the electrophoretic mobility by means of a zetameter (such as a Zetasizer 2000 or Zetasizer NanoS, Malvern Instruments, UK). The electrophoretic mobility is the converted into zeta potential values through the Smoluchowsky or Henry equation.

### DETAILED DESCRIPTION

Oil-In-Water Emulsion

This invention relates to an oil-in-water emulsion for intravitreal administration comprising an oil, an active ingredient, a mixture of at least two non-ionic surfactants, and water.

The oil is comprised in the dispersed phase of the emulsion, and water is comprised in the continuous phase of the emulsion. According to one embodiment, oil is the main component of the dispersed phase and water is the main component of the continuous phase. In this embodiment, according to terminology of common use in the art, it may be indicated that the oil “is” the dispersed phase and water “is” the continuous phase, although the dispersed phase and the continuous phase may actually comprise further components solubilized or suspended therein, e.g., active ingredients, surfactants, additives, etc.

In the invention, the emulsion is stabilized by means of a mixture of at least two non-ionic surfactants. Advantageously, the mixture of at least two non-ionic surfactants also helps obtaining appropriate droplet size in the emulsion.

Although some substances may possibly qualify simultaneously as an oil, an active ingredient and/or a non-ionic surfactant, in the emulsion of the invention the oil, the active ingredient and the at least two non-ionic surfactants refer to four different substances, i.e., in the emulsion of the invention the “oil” component cannot at the same time be the “active ingredient” component or a “non-ionic surfactant” component, etc. In others words, the emulsion of the invention systematically comprises at least four different substances other than water, at least one of them performing at least the function of the oil, another one of them performing at least the function of the active ingredient, and two others performing at least the function of the non-ionic surfactants. In others words, in the emulsion of the invention the oil, the active ingredient and the at least two non-ionic surfactants are distinct substances to one another.

According to one embodiment, the active ingredient is selected from:
antiallergenics such as sodium cromoglycate, antazoline, methapyrilene, chlorpheniramine, olopatadine, ketotifen, azelastine, emedastine, levocabastine, terfenadine, astemizole, loratadine, pyrilamine or prophenpyridamine;
synthetic glucocorticoids, mineralocorticoids and hormones forms deriving from the cholesterol metabolism such as progesterone, estrogens or androgenic hormones, e.g., testosterone DHEA and their derivatives;
anti-inflammatories such as cortisone, hydrocortisone, hydrocortisone acetate, dexamethasone, dexamethasone 21-phosphate, fluoroquinolone, medrysone, prednisone, methylprednisolone, prednisolone acetate, fluorometholone, triamcinolone, betamethasone, loteprednol, flumetasone (also known as flumethasone), mometasone, danazol, beclomethasone, difluprednate or triamcinolone acetonide;
non-steroidal anti-inflammatories such as salicylate, indomethacin, ibuprofen, diclofenac, flurbiprofen, 2-arylpropionic acids, N-arylanthranilic acids, oxicams, sulfonanilides, pyrazolidines derivatives, arylalkanoic acids, 3-benzolphenylacetic acids and their derivatives, piroxicam, or COX2 inhibitors such as rofecoxib, diclofenac, nimesulide or nepafenac;
anti-inflammatories and pro-resolving factors such as bioactive autacoid metabolites of arachidonic acid, e.g., lipoxin A4 (LXA4), LXB4, or their epimers (being the epi-lipoxins, 15-epi-LXA4 and 15-epi-LXB4);
antineoplastics such as carmustine, cisplatin, mitomycin or fluorouracil;
immunological drugs such as vaccines or immune stimulants;
insulin, calcitonin, parathyroid hormone and peptide and vasopressin hypothalamus releasing factor;
beta adrenergic blockers such as timolol maleate, levobunolol HCl, betaxolol HCl, timolol-base, betaxolol, atenolol, befunolol, metipranolol, forskolin, carteolol, epinephrine, dipivefrine (also known as dipivalyl epinephrine), oxonolol, acetazolamide-base or methazolamide;
cytokines, interleukins, prostaglandins such as latanoprost, bimatoprost or travoprost, antiprostaglandins, prostaglandin precursors, and growth factors such as epidermal growth factor, fibroblast growth factor, platelet derived growth factor, transforming growth factor beta, ciliary neurotrophic growth factor, glial derived neurotrophic factor, NGF, EPO or PLGF;
anti-angiogenic compounds such as VEGF inhibitors, VEGF soluble receptors, VEGF-traps, VEGF-antibodies, VEGF-traps or anti VEGF-siRNA;
antibodies or antibodies fragments, oligoaptamers, aptamers, and gene fragments such as oligonucleotides, plasmids, ribozymes, small interference RNA, nucleic acid fragments, peptides or antisense sequences;
immunomodulators such as natural or synthetic cyclosporines, cyclophosphamide (trade name Endoxan®), sirolimus, tacrolimus, thalidomide or tamoxifen;
secretagogues such as pilocarpine or cevimeline (trade name Evoxac®);
mucin secretagogues such as 15(S)-HETE or ecabet;
antithrombotic and vasodilator agents such as rtPA, urokinase, plasmin, or nitric oxide donors;
androgen mimetics, flaxseed oil supplements, agonists of adenosine A3 receptor, and squalene;
antioxidants such as lutein or vitamins, especially vitamin A;
inhibitors of carbonic anhydrase such as brinzolamide, dorzolamide, acetazolamide, methazolamide, or dichlorphenamide;
sympathomimetics such as brimonidine, apraclonidine, dipivefrine, or epinephrine;
parasympathomimetics such as pilocarpine;
cholinesterase inhibitors such as physostigmine or echothiophate;
antivirals such as idoxuridine, trifluorothymidine (also known as trifluridine), acyclovir, valaciclovir, ganciclovir, cidofovir or interferon;
antibiotics such as aminoglycosides, carbacephems, carbapenems, cephalosporins, glycopeptides, penicillins, polypeptides, quinolones, sulfonamides, tetracyclines, chlortetracycline, bacitracin, neomycin, polymyxin, gramicidin, cephalexin, oxytetracycline, chloramphenicol, kanamycin, rifampicin, tobramycin, gentamycin, ciprofloxacin, erythromycin, ceftazidime, vancomycin or imipenem;
antifungals such as polyene antibiotics, azole derivatives, imidazole, triazole, allylamines, amphotericin B or miconazole;
antibacterials such as sulfonamides, sulfadiazine, sulfacetamide, sulfamethizole and sulfisoxazole, nitrofurazone or sodium propionate;
derivatives thereof; prodrugs thereof; and acceptable salts thereof.

According to one embodiment, the active ingredient is selected from anti-inflammatories such as steroids, steroid derivatives, steroid prodrugs or steroid acceptable salts.

According to one embodiment, the active ingredient is lipophilic. According to one embodiment, the active ingredient has a log P ranging from about 5 to about 15, preferably ranging from about 6 to about 14, more preferably ranging from about 7 to about 13, furthermore preferably ranging from about 8 to about 12. The log P of dexamethasone palmitate calculated by XLogP3 method is 9.8 (about 10), so that dexamethasone palmitate is highly lipophilic.

According to one embodiment, the active ingredient is a prodrug. According to one embodiment, the active ingredient is a long chain ester of a drug. Long chain esters of drugs are commonly used prodrugs. In one embodiment, the active ingredient is a C₁₀-C₂₁ ester. In one embodiment, the active ingredient is a C₁₀-C₁₈ ester, preferably a C₁₂-C₁₆ ester, more preferably a C₁₄ ester. The release of the drug from an ester prodrug occurs via an enzymatic process in retina and/or choroid. The prodrug comprises the long chain ester function that can be cleaved by an enzyme present in the ocular tissue. In the invention, the metabolization of the drug typically occurs in retina and/or choroid after intravitreal administration. The cleaving enzymes may for example be esterases (e.g., pseudocholinesterase or acetylcholine esterase), oxidoreductases, transferases, lyases, isomerases, ligases, hydrolases, phosphatases, proteases or peptidases. Typically, the release of the drug from the ester prodrug occurs via the action of one or more esterases.

According to one embodiment, the active ingredient is a long chain ester of a steroid. Long chain esters of steroids are well-known prodrugs of steroids. In one embodiment, the active ingredient is a C₁₀-C₂₁ ester of a steroid. In one embodiment, the active ingredient is a C₁₀-C₁₈ ester of a steroid, preferably a C₁₂-C₁₆ ester of a steroid, more preferably a C₁₄ ester of a steroid. In one embodiment, the steroid is a corticosteroid. In one embodiment, the corticosteroid is selected from alclometasone, amcinonide, amcinafal, amcinafide, beclomethasone (also known as beclometasone or beclometasone dipropionate), betamethasone, chloroprednisone, clobetasone, clocortolone, cortodoxone, cortisol (also known as hydrocortisone), ciclesonide, descinolone, desonide, deflazacort, diflorasone, difluprednate, desoximetasone, dexamethasone, dichlorisone, fluazacort, flucloronide, fludrocortisone, flumetasone (also known as flumethasone), flunisolide, fluocinonide, fluocinolone, fluocortolone, fluclorolone, fludroxycortide (also known as flurandrenolone or flurandrenolide), fluorocortisone, fluorometholone, fluperolone, fluprednisolone, fluticasone, hydrocortamate, loteprendol, medrysone, meprednisone, methylprednisolone, mometasone, paramethasone, prednisolone, rimexolone, triamcinolone and pharmaceutically acceptable salts thereof. In one embodiment, the corticosteroid is selected from prednisolone, fluorometholone, dexamethasone, rimexolone, medrysone and pharmaceutically acceptable salts thereof. In one embodiment, the corticosteroid is dexamethasone or a pharmaceutically acceptable salt thereof. In one embodiment, the long chain ester of dexamethasone is selected from dexamethasone caprate (a C₁₀-ester of dexamethasone), dexamethasone laurate (a C₁₂-ester of dexamethasone), dexamethasone myristate (a C₁₄-ester of dexamethasone), dexamethasone palmitate (a C₁₆-ester of dexamethasone) and dexamethasone stearate (a C₁₈-ester of dexamethasone). In one embodiment, the lipophilic long chain ester of dexamethasone is selected from dexamethasone laurate, dexamethasone myristate and dexamethasone palmitate. In one embodiment, the lipophilic long chain ester of dexamethasone is dexamethasone palmitate.

According to one embodiment, the active ingredient is the only active ingredient in the composition. In one embodiment, the active ingredient is used in combination with at least another active ingredient. According to one embodiment, the emulsion comprises the active ingredient in an amount ranging from about 0.001% to about 10% w/w, preferably ranging from about 0.01% to about 7.5% w/w, more preferably ranging from about 0.1% to about 5% w/w. In one embodiment, the emulsion comprises the active ingredient in an amount ranging from about 0.1 to about 5% w/w, preferably ranging from about 0.25 to about 2.5% w/w, more preferably ranging from about 0.5 to about 1% w/w. “w/w” means “in weight of the total weight of the emulsion”. According to one preferred embodiment, the active ingredient is comprised in the oil, and therefore is comprised in the dispersed phase. According to another embodiment, the active ingredient is comprised in the water, and therefore is comprised in the continuous phase.

According to one embodiment, the oil comprises an oil selected from triglyceride oils such as short chain triglycerides (C₁-C₅ triglycerides), medium chain triglycerides (C₆-C₁₂ triglycerides), long chain triglycerides (C₁₃-C₂₁ triglycerides) or very long chain triglycerides (C₂₂ or more, typically C₂₂-C₃₄ triglycerides); mineral oils such as petrolatum, liquid paraffin, heavy mineral oil or light mineral oil; vegetable oils such as castor oil, corn oil, olive oil, soybean oil, sesame oil, cotton seed oil or sweet almond oil; fatty acids; isopropyl myristate; oily fatty alcohols; sorbitol esters and/or sorbitol fatty acids; oily sucrose esters; and mixtures thereof. In one embodiment, the oil comprises mineral oils, preferably a mixture of light mineral oil and heavy mineral oil.

In one embodiment, the oil comprises triglyceride oils. In the invention, triglycerides may have identical and different fatty acid chains. In one embodiment, the oil comprises, substantially consists in, or consists in, medium chain triglycerides (MCT). Medium chain triglycerides (MCT) may be obtained for example from palm kernel oils or coconut oils. Medium chain triglycerides (MCT) have a density ranging from 0.93 to 0.96.

Without being bound by any theory, the Applicant believes that the use of triglyceride when formulating lipophilic active ingredients such as long-chain ester prodrugs may facilitate the solubilization of the active ingredient, the formation of droplets of controlled size, the stabilization of the droplets in the emulsion or in the vitreous body, the controlled and/or sustained delivery of the active ingredient to the eye and/or the prevention of troubles of vision.

According to one embodiment, the composition comprises the oil in an amount ranging from about 0.01% to about 50% w/w, preferably ranging from about 0.1% to about 25% w/w, more preferably ranging from about 0.5% to about 15% w/w. In one embodiment, the composition comprises the oil in an amount ranging from about 0.5% to about 15% w/w, preferably ranging from about 0.75% to about 10% w/w, more preferably ranging from about 1% to about 5% w/w. In one embodiment, the composition comprises the oil in an amount ranging from about 0.01% to about 15% w/w, preferably ranging from about 0.1% to about 5% w/w, more preferably ranging from about 0.3% to about 3% w/w. “w/w” means “in weight of the total weight of the emulsion”.

According to one embodiment, the non-ionic surfactants comprise surfactants selected from poloxamers such as poloxamer 282 or poloxamer 188 or Pluronic® F-68LF or Lutrol® F68; polyoxyethylene castor oils (polyethoxylated castor oils) such as Cremophor EL® or Cremophor RH®; polyoxyethylene alkyl ethers; polyoxyethylene fatty acid esters such as Emulphor®; polyethylene glycol (15)-hydroxystearate (trade name Solutol®); polysorbates such as polysorbate 20 (trade name Tween® 20) or polysorbate 80 (trade name Tween® 80); polyoxyethylene sorbitan fatty acid esters; polyoxyethylene stearates; tyloxapol; sorbitan esters such as Span™ 20, Span™ 40, Span™ 60, Span™ 65, Span™ 80 or Span™ 85; vitamin E derivatives such as D-α-tocopheryl polyethylene glycol succinate (“TPGS” or “Vitamin E-TPGS”) and mixtures thereof. In one embodiment, the non-ionic surfactants are selected from polyoxyethylene castor oils and sorbitan esters. In one specific embodiment, the mixture of two non-ionic surfactants consists in a mixture of at least one polyoxyethylene castor oil and at least one sorbitan ester. In one further specific embodiment, the polyoxyethylene castor oil is polyoxyl 35 castor oil (CAS [61791-12-6] or [63393-92-0]; also known as PEG-35 castor oil or as polyoxyl-35 castor oil or as macrogolglycerol ricinoleate 35), e.g., commercial product Cremophor EL® (also known as Kolliphor EL®). In one further specific embodiment, the sorbitan ester is sorbitan monolaurate, e.g., commercial product Span™ 20. In one embodiment, the non-ionic surfactants are selected from polyoxyethylene castor oils and polysorbates. In one specific embodiment, the mixture of two non-ionic surfactants consists in a mixture of at least one polyoxyethylene castor oil and at least one polysorbate. In one further specific embodiment, the polyoxyethylene castor oil is polyoxyl 35 castor oil (CAS [61791-12-6] or [63393-92-0]; also known as PEG-35 castor oil or as polyoxyl-35 castor oil or as macrogolglycerol ricinoleate 35), e.g., commercial product Cremophor EL® (also known as Kolliphor EL®). In one further specific embodiment, the polysorbate is polysorbate 20, e.g., commercial product Tween® 20.

Without being bound by any theory, the Applicant believes that the use of polyoxyethylene castor oils and sorbitan esters and/or polysorbates as surfactants when formulating active ingredients may facilitate the solubilization of the active ingredient, the formation of droplets of controlled size, the stabilization of the droplets in the emulsion or in the vitreous body, the controlled and/or sustained delivery of the active ingredient to the eye and/or the prevention of troubles of vision.

In one embodiment, the non-ionic surfactants have an HLB of 10 or more, 11 or more, 12 or more, 13 or more, or 14 or more. Examples of such surfactants may include polyoxyethylene castor oils or sorbitan esters.

According to one embodiment, the emulsion further comprises a cationic surfactant and/or an anionic surfactant. In one embodiment, the anionic surfactant is selected from anionic phospholipids such as lecithins, docusate sodium, emulsifying wax BP, sodium lauryl sulfate and a mixture thereof. In one embodiment, the cationic surfactant is selected from quaternary ammonium compounds such as benzalkonium chloride (BAK), cetalkonium chloride (CKC), benzethonium chloride, cetrimide, cationic lipids, oleylamine, stearylamine, DOTAP (N-[1-(2,3-dioleoyloxy)propyl]-N,N,N trimethylammonium) chloride, DOPE (dioleoylphosphatidylethanolamine), poly(ethylenimine) (PEI), poly-L-lysine (PLL) and a mixture thereof.

According to one embodiment, the emulsion comprises the surfactants in an amount ranging from 0.001% to 25% w/w, preferably ranging from about 0.01% to about 15% w/w, more preferably ranging from about 0.2% to about 10% w/w. In one embodiment, the emulsion comprises the surfactants in an amount ranging from about 0.2% to about 10% w/w, preferably ranging from about 0.5% to about 5% w/w, more preferably ranging from about 1% to about 3% w/w. The preceding ranges may apply either to the total amount of surfactants (anionic, cationic or non-ionic) or to the total amount of non-ionic surfactants. “w/w” means “in weight of the total weight of the emulsion”. According to one embodiment, the emulsion comprises the two non-ionic surfactants in a relative ratio in weight ranging from about 10/90 to about 90/10, preferably from about 15/85 to about 85/15, more preferably from about 25/75 to about 75/25, furthermore preferably from about 50/50 to about 75/25. In one embodiment, the emulsion comprises the two non-ionic surfactants in a relative ratio in weight of about 10/90, about 15/85, about 25/75, about 50/50, about 75/25, about 85/15 or about 90/10. In one embodiment, the emulsion comprises the two non-ionic surfactants in a relative ratio in weight of about 25/75, about 50/50 or about 75/25. According to one embodiment, the emulsion has a ratio in weight total amount of oils/total amount of surfactants ranging from about 0.1 to about 5, preferably ranging from about 0.2 to about 4, more preferably about 0.5 to about 3. In one embodiment, the emulsion has a ratio in weight total amount of oils/total amount of surfactants ranging from about 1 to about 2.5, preferably ranging from about 1.5 to about 2, more preferably more preferably about 1.7.

According to one embodiment, the water is selected from tap water, saline solution (saline), distilled water and ultrapure water. The water may for example be water for injection (also known as aqua ad iniectabilia or aqua ad injectionem).

According to one embodiment, the emulsion comprises one or more additive(s) such as antioxidants, antimicrobials, buffers, chelating agents, osmotic agents, pH adjusters, preservatives, solubilizers, stabilizers, thickening agents, viscosity modulator agents or colorants. In one embodiment, the emulsion comprises at least one osmotic agent selected from glycerol (glycerin), mannitol, sorbitol, xylitol, propylene glycol, sodium chloride, potassium chloride, magnesium chloride, calcium chloride, and mixtures thereof. In one embodiment, the osmotic agent is selected from glycerol, mannitol, sorbitol and mixtures thereof. In one embodiment, the osmotic agent comprises glycerol. In one embodiment, the emulsion comprises the osmotic agent in an amount ranging from 0.1% to 20% w/w, preferably ranging from about 0.25% to about 10% w/w, more preferably ranging from about 0.5% to about 5% w/w. “w/w” means “in weight of the total weight of the emulsion”. In one embodiment, the composition is free of preservative agent, i.e., it is “preservative-free”. In another embodiment, the composition comprises at least one preservative agent selected from benzyl alcohol, boric acid, chlorhexidine, quaternary ammonium salts such as benzalkonium chloride (BAK), mercury salts, thiomersal, and salts thereof, and mixtures thereof.

In one embodiment, the emulsion comprises medium chain triglycerides (MCT), dexamethasone palmitate, polyoxyl 35 castor oil, sorbitan monolaurate, glycerol and water.

According to one embodiment, the emulsion is free of azithromycin. In one embodiment, the emulsion is free of antibiotics. According to one embodiment, the emulsion is free of latanoprost. In one embodiment, the emulsion is free of prostaglandins. According to one embodiment, the emulsion is free of isopropyl myristate. In one embodiment, the emulsion is free of fatty esters. According to one embodiment, the emulsion is free of cationic surfactants. According to one embodiment, the emulsion is free of anionic surfactants. According to one embodiment, the emulsion is free of phospholipids such as lecithins (for example Epikuron™, Ovothin™ or Lipoid™, especially Lipoid E80). According to one embodiment, the emulsion is free of poloxamers such as Pluronic® F-68 (Lutrol F68). According to one embodiment, the emulsion is free of tyloxapol. According to one embodiment, the emulsion is free of polysorbate 20 (trade name Tween® 20). According to one embodiment, the emulsion is free of polysorbate 80 (trade name Tween® 80). In one embodiment, the emulsion is free of polysorbates. In the presence disclosure, recitations such as “is free of” and “does not contain any” have the same meaning and refer to the absence of a stated compound in a composition, the absence being considered according to purity standards and analytical methods of common use in the art, especially of common use in the ophthalmic field.

According to one embodiment, the emulsion is an anionic emulsion, i.e., an emulsion having a negative zeta potential, typically a zeta potential lower than or equal to −10 mV. Inclusion of anionic surfactants in the emulsion (as described hereinabove) is a way to render it anionic by conferring it a negative charge. In one embodiment, the anionic emulsion has a zeta potential lower than or equal to about −15 mV, preferably lower than or equal to about −20 mV, more preferably lower than or equal to about −25 mV, furthermore preferably lower than or equal to about −30 mV. According to one embodiment, the emulsion is a cationic emulsion, i.e., an emulsion having a positive zeta potential, typically a zeta potential higher than or equal to 10 mV. In one embodiment, the cationic emulsion has a zeta potential higher than or equal to about 15 mV, preferably higher than or equal to about 20 mV, more preferably higher than or equal to about 25 mV, furthermore preferably higher than or equal to about 30 mV. Inclusion of cationic surfactants in the emulsion (as described hereinabove) is a way to render it cationic by conferring it a positive charge. According to one embodiment, the emulsion is a non-ionic emulsion, i.e., an emulsion having a zeta potential close to zero, typically a zeta potential between 10 mV and −10 mV (i.e., not including 10 mV and −10 mV values).

According to one embodiment, the emulsion is anionic and free of anionic surfactants. The Applicant surprisingly found that the use of a mixture of at least two non-ionic surfactants may sometime lead to anionic emulsions. Without being limited by any theory, the Applicant thinks that during the manufacturing process, the emulsion may release negatively charged ingredients. In one embodiment, the anionic emulsion is made of starting components which are not negatively charged, especially the starting materials for the manufacturing of the emulsion do not include any anionic surfactants.

Anionic emulsions may be preferred for intravitreal administration because cationic emulsions may cause inflammation when administered intraocularly.

According to one embodiment, the emulsion has a droplet size ranging from about 50 to about 250 nm; preferably ranging from about 100 to about 200 nm; more preferably ranging from about 110 to about 175 nm. In one embodiment, the emulsion has a droplet size ranging from about 100 to about 200 nm; preferably ranging from about 110 to about 175 nm, more preferably ranging from about 120 nm to about 160 nm. In one embodiment, the emulsion has a droplet size of about 115 nm, about 120 nm, about 125 nm or about 130 nm. In one embodiment, the emulsion has a droplet size of about 145 nm, about 150 nm, about 155 nm, about 160 nm, about 165 nm or about 170 nm. In one embodiment, the emulsion is not a microemulsion. In one embodiment, the emulsion has a droplet size higher than or equal to about 75 nm, about 100 nm, about 110 nm or about 120 nm. In one embodiment, the emulsion has a droplet size lower than or equal to about 225 nm, about 200 nm, about 175 nm or about 160 nm.

Without being bound by any theory, the Applicant believes that the use of emulsions having a droplet size not lower than about 100 nm when formulating active ingredients may facilitate the stabilization of the droplets in the emulsion or in the vitreous body and/or the controlled and/or sustained delivery of the active ingredient to the eye. Without being bound by any theory, the Applicant believes that the use of emulsions having a droplet size not higher than about 200 nm when formulating active ingredients may facilitate the stabilization of the droplets in the emulsion or in the vitreous body, the controlled and/or sustained delivery of the active ingredient to the eye and/or the prevention of troubles of vision.

The emulsion of the invention leads to translucence of vitreous gel after intravitreal administration. The visual trouble of emulsions following intravitreal administration depends on the turbidity of the emulsion and is related to the opacity of the emulsion. Therefore, emulsions for intravitreal injection should be of the lowest turbidity (i.e., the lowest light absorbance or the highest light transmittance), in other words they should be as translucent as possible. Light transmittance/absorbance of 50% is generally considered in the art as the lower vision limit.

Translucence of the vitreous gel after intravitreal administration of an emulsion may be simulated in vitro by diluting a volume of the emulsion (corresponding to the injected volume) in about 4 mL (i.e., the average volume of the vitreous gel in adulthood) of an aqueous solution such as water, saline, etc. Dilution into the vitreous after intravitreal injection is typically from about 1:100 (i.e., a ratio in volume emulsion/water of about 0.01) to about 1:500 (i.e., a ratio in volume emulsion/water of about 0.002), preferably about 1:100. According to one embodiment, the emulsion has a light transmittance after being diluted in water in a ratio in volume emulsion/water of about 0.005 (corresponding for example to 20 μL in 4 mL) ranging from about 50% to about 100%, preferably ranging from about 75% to about 100%, more preferably ranging from about 80% to about 100%, further more preferably ranging from about 85% to about 100%. According to one embodiment, the emulsion has a light transmittance after being diluted in water in a ratio in volume emulsion/water of about 0.01 (corresponding for example to 10 μL in 1 mL or 40 μL in 4 mL or 80 μL in 8 mL) ranging from about 50% to about 100%, preferably ranging from about 70% to about 100%, more preferably ranging from about 75% to about 100%, further more preferably ranging from about 80% to about 100%. According to one embodiment, the emulsion has a light transmittance after being diluted in water in a ratio in volume emulsion/water of about 0.0125 (corresponding for example to 50 μL in 4 mL) ranging from about 50% to about 100%, preferably ranging from about 70% to about 100%, more preferably ranging from about 75% to about 100%, further more preferably ranging from about 80% to about 100%. According to one embodiment, the emulsion has a light transmittance after being diluted in water in a ratio in volume emulsion/water of about 0.025 (corresponding for example to 100 μL in 4 mL) ranging from about 50% to about 100%, preferably ranging from about 65% to about 100%, more preferably ranging from about 70% to about 100%, further more preferably ranging from about 75% to about 100%.

In one embodiment, the invention relates to an oil-in-water emulsion for intravitreal administration comprising: from about 0.01 to about 50% w/w of an oil being a triglyceride oil, from about 0.001 to about 10% w/w of a lipophilic active ingredient comprised in said oil, from about 0.001 to about 25% w/w of a mixture of at least two non-ionic surfactants, and water; wherein said emulsion has a droplet size ranging from about 100 to about 200 nm; and wherein said emulsion has a light transmittance after being diluted in water in a ratio in volume emulsion/water of about 0.01 ranging from about 70% to about 100%.

Translucence of the vitreous gel after intravitreal administration of an emulsion may also be simulated in vivo by intravitreal injection into the eye of a model animal, e g, a rabbit.

The emulsion of the invention is advantageously highly translucent, even before intravitreal administration. According to one embodiment, the emulsion has a light transmittance ranging from about 50% to about 100%, preferably ranging from about 60% to about 100%, more preferably ranging from about 70% to about 100%, further more preferably ranging from about 80% to about 100%. In one embodiment, the emulsion has a light transmittance ranging from about 80% to about 100%, preferably ranging from about 85% to about 100%, more preferably ranging from about 90% to about 100%, further more preferably ranging from about 95% to about 100%.

The emulsion of the invention is advantageously stable, i.e., they can be stored overtime without destabilization of the emulsion and/or without degradation of the active ingredient. According to one embodiment, the emulsion can be stored for 3 months, preferably 6 months, more preferably 1 year.

The emulsion of the invention is advantageously sterilisable by methods known in the art, in accordance with safety requirements in the ophthalmic field. Especially, the emulsion keeps its structure and/or properties when sterilised. For example, the emulsion may be sterilisable by steam sterilisation by autoclave at about 120° C. during 10 to 30 min.

According to one embodiment, the emulsion is not a self-emulsifying oil. According to one embodiment, the emulsion is not comprised in a self-emulsifying oil. According to one embodiment, the emulsion is not a self-emulsifying drug delivery system (SEDDS). According to one embodiment, the emulsion is not comprised in a self-emulsifying drug delivery system (SEDDS).

Medical Uses

This invention also relates to an emulsion according to the invention, as described hereinabove, for use as a medicament.

This invention also relates to an emulsion according to the invention, as described hereinabove, for use in the treatment of an eye disease or condition. According to one embodiment, the eye disease or condition is a disease or condition of the posterior segment of the eye, especially of the back of the eye (e.g., of retina). In one embodiment, the eye disease or condition is selected from uveitis, macular edema such as diabetic macular edema (DME), macular degeneration such as age-related macular degeneration (AMD or ARMD), retinal detachment, ocular tumors, bacterial infections, fungal infections, viral infections, multifocal choroiditis, diabetic retinopathy, proliferative vitreoretinopathy (PVR), sympathetic ophthalmia, Vogt-Koyanagi-Harada (VKH) syndrome, histoplasmosis, uveal diffusion and vascular occlusion. In one embodiment, the eye disease or condition is diabetic macular edema (DME). In one embodiment, the eye disease or condition is age-related macular degeneration (AMD).

According to one embodiment, the emulsion is a pharmaceutical composition.

According to one embodiment, the use of the emulsion comprises a step of intravitreal administration of the emulsion. In one embodiment, the emulsion is intravitreally administered in an amount ranging from about 1 to about 500 μL, preferably ranging from about 5 to about 250 μL, more preferably ranging from about 10 to about 100 μL, furthermore preferably ranging from about 25 to about 50 μL. In one embodiment, the emulsion is intravitreally administered in an amount of about 20 μL, about 25 μL, about 30 μL, about 35 μL, about 40 μL, about 45 μL or about 50 μL. In one embodiment, the emulsion is intravitreally administered by means of an implantable device. In one embodiment, the emulsion is intravitreally administered by means of a syringe. In one embodiment, the emulsion is intravitreally injected, i.e., the intravitreal administration is intravitreal injection.

According to one embodiment, the emulsion is not for topical use, i.e., administration to the surface of the eye, such as onto the cornea. According to one embodiment, the use of the emulsion does not comprise a step of topical administration of the emulsion. According to one embodiment, the emulsion is not for use in the treatment of dry eye.

Advantageously, when the active ingredient is a prodrug and the emulsion is intravitreally administered, the corresponding drug is not present in the vitreous body 3 or 6 months after the intravitreal administration, but the corresponding drug is present in other parts of the posterior segment of the eye (such as the retina or the choroid) after the intravitreal administration for at least 3 months, preferably for at least 6 months. Advantageously, when the active ingredient is a prodrug and the emulsion is intravitreally administered, the prodrug is present in the vitreous body after the intravitreal administration for at least 3 months, preferably for at least 6 months.

This invention also relates to the use of an emulsion according to the invention, as described hereinabove, in the manufacture of a medicament for the treatment of eye diseases or conditions. According to one embodiment, the medicament is administered by intravitreal administration, preferably intravitreal injection. This invention also relates to a method for the treatment of eye diseases or conditions in a subject in need thereof, comprising a step of administering to the subject a therapeutically effective amount of an emulsion according to the invention, as described hereinabove. According to one embodiment, the step of administering comprises a step of intravitreal administration, preferably intravitreal injection, of the emulsion to the subject.

Devices

This invention also relates to an implantable device comprising the oil-in-water emulsion according to the invention, as described hereinabove. According to one embodiment, the implantable device is biodegradable.

This invention also relates to a device for intravitreal injection comprising the oil-in-water emulsion according to the invention, as described hereinabove. According to an embodiment, the device for intravitreal injection is a syringe. In one embodiment, the device is a prefilled syringe. In one embodiment, the syringe has a 22 to 33-gauge needle, preferably a 25 to 30-gauge needle.

According to an embodiment, the implantable device or the device for intravitreal injection comprises an amount of the emulsion ranging from about 1 to about 500 μL, preferably ranging from about 5 to about 250 μL, more preferably ranging from about 10 to about 100 μL. In one embodiment, the device comprises of about 20 μL, about 25 μL, about 30 μL, about 35 μL, about 40 μL, about 45 μL or about 50 μL of the emulsion.

According to one embodiment, the emulsion is packaged in glass vials. According to one embodiment, the emulsion is packaged in unitary dose forms. According to another embodiment, the emulsion is packaged in multi-dose containers.

According to one embodiment, the emulsion is not eye drops. According to one embodiment, the emulsion is not comprised in eye drops. According to one embodiment, the emulsion is not in the form of eye drops.

Manufacturing Process

This invention also relates to a process for manufacturing an oil-in-water emulsion according to the invention, as described hereinabove.

According to one embodiment, the process comprises the steps of:
stirring together the components of the dispersed (oil) phase;
stirring together the components of the continuous (aqueous) phase;
optionally heating both phases were heated between about 50 and about 80° C.;
adding the aqueous phase in the oily phase;
optionally heating the mixture between about 60 and about 90° C.;
decreasing the droplet size, preferably by high shear mixing during 1 to 10 min;
optionally cooling down between about 10 and about 30° C.; and
homogenizing, preferably on a microfluidizer, thereby obtaining the final emulsion.

According to another embodiment, the process comprises the steps of:
stirring together the components of the dispersed (oil) phase;
stirring together the components of the continuous (aqueous) phase;
optionally heating both phases were heated between about 50 and about 80° C.;
adding the aqueous phase in the oily phase;
optionally heating the mixture between about 60 and about 90° C.;
decreasing the droplet size, preferably by high shear mixing during 1 to 10 min;
optionally cooling down between about 1 to about 3 hours.

In one embodiment, the stirring is magnetic stirring.

### BRIEF DESCRIPTION OF THE DRAWINGS

FIG. 1 is a histogram showing the amounts of dexamethasone (DXM) in the vitreous body 6 months after intraocular injection for the 3 tested doses of compositions Z31EM588 and Z31EM589.

FIG. 2 is a histogram showing the amounts of dexamethasone (DXM) in the retina 6 months after intraocular injection for the 3 tested doses of the compositions Z31EM588 and Z31EM589.

FIG. 3 is a histogram showing the amounts of dexamethasone (DXM) in the choroid 6 months after intraocular injection for the 3 tested doses of the compositions Z31EM588 and Z31EM589.

FIG. 4 is a histogram showing the amounts of dexamethasone palmitate (DXP) in the vitreous body 6 months after intraocular injection for the 3 tested doses of the compositions Z31EM588 and Z31EM589.

FIG. 5 is a histogram showing the amounts of dexamethasone palmitate (DXP) in the retina 6 months after intraocular injection for the 3 tested doses of the compositions Z31EM588 and Z31EM589.

FIG. 6 is a histogram showing the amounts of dexamethasone palmitate (DXP) in the choroid 6 months after intraocular injection for the 3 tested doses of the compositions Z31EM588 and Z31EM589.

FIG. 7 is a graph showing the droplet size of emulsions having different oil contents (3, 6.5 and 10% w/w) with different Cremophor EL®/Span™ 20 ratios, without autoclaving.

FIG. 8 is a graph showing the droplet size of emulsions having different oil contents (3, 6.5 and 10% w/w) with different Cremophor EL®/Span™ 20 ratios, after autoclaving.

### EXAMPLES

The present invention is further illustrated by the following examples.

### Example 1: Oil-In-Water Emulsions

Materials and Methods

Materials: The substances used in the preparation of the emulsions were purchased from commercial providers and used without further purification.

Methods—Manufacture of the emulsions Z31EM090, Z31EM588, Z31EM589, Z01EM1515 and Z01EM1516: Two hundred grams of each emulsion were prepared as described hereinafter: Weighing of the oil phase components in a beaker; Dissolution of the oily phase components under magnetic stirring (200 rpm) and slight heating (50° C.); Heating of the oily phase to 65° C. under magnetic stirring (200 rpm). Parallelly, Weighing of the aqueous phase components in a beaker; Dissolution of the aqueous phase components under magnetic stirring (200 rpm) and slight heating (50° C.); Heating of the aqueous phase to 65° C. under magnetic stirring (200 rpm). Then, Addition of aqueous phase in the oily phase at 65° C. under magnetic stirring (300-400 rpm); Heating of the coarse emulsion to 75° C. under magnetic stirring (300-400 rpm); Dispersion with Polytron homogenizer (Polytron® PT 6100, Kinematica): 5 min at 16000 rpm; Cooling of the emulsion to 25° C. using an ice bath; Continuous homogenization: 15000 psi 10 min (for 100 mL) or 15 min (for 150 mL) with Emulsiflex homogenizer (Avestin® Emulsiflex C3); Cooling of the emulsion to 25° C. using an ice bath; pH measurement and adjustment at pH 7.0 with NaOH 0.1 M. The emulsions were then distributed in glass vials and sterilized by autoclave during 20 min at 121° C. Manufacture of the emulsions Z31EM433 and Z31EM434: Two hundred grams of each emulsion were prepared as described hereinafter: Weighing of the oil phase components in a beaker; Dissolution of the oily phase components under magnetic stirring (200 rpm) and slight heating (50° C.); Heating of the oily phase to 65° C. under magnetic stirring (200 rpm). Parallelly, Weighing of the aqueous phase components in a beaker; Dissolution of the aqueous phase components under magnetic stirring (200 rpm) and slight heating (50° C.); Heating of the aqueous phase to 65° C. under magnetic stirring (200 rpm). Then, Addition of aqueous phase in the oily phase at 65° C. under magnetic stirring (300-400 rpm); Cooling of the emulsion under stirring during 2 hours to 25° C. using an ice bath; Storage one night (12 hours) at room temperature. The emulsions were then distributed in glass vials and sterilized by autoclave during 20 min at 121° C. Droplet size: Droplet size was measured after a 1/20 dilution (50 μL of emulsion in water q.s. 1 mL) using a Zetasizer NanoS (Malvern Instruments, UK). Turbidity: Turbidity (light transmittance in %) was measured with a Turbiscan Beckman Coulter (United States) on 8 mL of sample after a 1/100 dilution in water (80 μL of sample in 8 mL of water). Zeta potential: Zeta potential was measured with a Zetasizer NanoS (Malvern Instruments, UK) after a 1/250 dilution (80 μL of emulsion in water q.s. 20 mL) in clear disposable zeta cells. pH: pH was measured with a Seven Multi Mettler Toledo XP205DR without undergoing any dilution. Osmolality: The osmolality was measured on 100 μL of sample without undergoing any dilution.

Results

The emulsions with the compositions as shown in Table 1 have been prepared.

TABLE 1

| | Z31EM588 | Z31EM589 | |
|---|---|---|---|
| | (% w/w) | (% w/w) | |
| Dexamethasone | 0.8 | 0.8 | |
| palmitate (DXP) | | | |
| MCT | 3.0 | 3.0 | |
| Cremophor EL® | 1.35 | 0.9 | |
| Span™ 20 | 0.45 | 0.9 | |
| Glycerol | 2.0 | 2.0 | |
| Water | Qs 100 | Qs 100 | |
| | Z01EM1515 | Z01EM1516 | |
| | (% w/w) | (% w/w) | |
| MCT | 3.0 | 3.0 | |
| Cremophor EL® | 1.35 | 0.9 | |
| Span™ 20 | 0.45 | 0.9 | |
| Glycerol | 2.0 | 2.0 | |
| Water | Qs 100 | Qs 100 | |
| | Z31EM090 | Z31EM433 | Z31EM434 |
| | (% w/w) | (% w/w) | (% w/w) |
| DXP | 3.2 | 0.8 | 0.8 |
| MCT | 14.0 | 2.0 | 3.0 |
| Cremophor EL® | — | 2.2 | 3.0 |
| Span™ 20 | — | — | — |
| Lipoid™ | 2.1 | — | — |
| Glycerol | 2.0 | 2.2 | 2.2 |
| Water | Qs 100 | Qs 100 | Qs 100 |

The emulsions have the properties as detailed in Table 2.

TABLE 2

| | Z31EM588 | Z31EM589 | |
|---|---|---|---|
| Droplet size | 122 | 161 | |
| (peak) (nm) | | | |
| Polydispersity | 0.100 | 0.148 | |
| index (PdI) | | | |
| Turbidity | 89 | 86 | |
| (% transmittance) | | | |
| Zeta potential | -30.6 | -37.4 | |
| (mV) [t₀] | | | |
| pH (t₀) | 5.8 | 6.0 | |
| Osmolality | 262 | 265 | |
| (mOsm/kg) | | | |
| | Z01EM1515 | Z01EM1516 | |
| Droplet size | 111 | 117 | |
| (peak) (nm) | | | |
| Polydispersity | 0.034 | 0.105 | |
| index (PdI) | | | |
| Turbidity | 91 | 92 | |
| (% transmittance) | | | |
| Zeta potential | -20.3 | -29.1 | |
| (mV) [t₀] | | | |
| pH (t₀) | 6.0 | 6.1 | |
| Osmolality | 261 | 263 | |
| (mOsm/kg) | | | |
| | Z31EM090 | Z31EM433 | |
| Droplet size (nm) | 242 | 77 | 74 |
| Polydispersity | 0.144 | 0.118 | 0.133 |
| index (PdI) | | | |
| Turbidity | 15% | 94% | 93% |
| % transmittance) | | | |
| Zeta potential | -40.7 | -7.0 | -6.2 |
| (mV) [t₀] | | | |
| pH (t₀) | 8.1 | 6.6 | 6.7 |
| Osmolality | 330 | 275 | 284 |
| (mOsm/kg) | | | |

Therefore, the compositions according to the invention comprising two non-ionic surfactants (Z31EM588 and Z31EM589 comprising an active ingredient and Z01EM1515 and Z01EM1516 not comprising any active ingredient) have a droplet size ranging from 100 to about 200 nm, whereas comparative emulsions comprising only one non-ionic surfactant (Z31EM433 and Z31EM434) have a droplet size far lower than 100 nm. The compositions according to the invention are also more translucent than comparative emulsions.

Comparative emulsion Z31EM090 comprising high amounts of oil (14% w/w MCT) in presence of only one surfactant has a droplet size higher than 200 nm. Moreover, due to its very low translucency, it is not suitable for intravitreal administration because it will cause vision issues such as burring (cf. Example 2 below).

### Example 2: Turbidity Assay: Intravitreal Injection Model

Materials and Methods

The emulsions were prepared as described in Example 1 hereinabove.

Turbidity method: The tested emulsions were diluted in water at 40, 80, 100, 160 and 200 μL in 8 mL. Then the turbidity of each sample (% light transmittance) was measured with a Turbiscan Beckman Coulter (United States).

Results

The results of the turbidity study are presented on Table 3.

TABLE 3

| Dilution in | Eq. dilution in | | | |
|---|---|---|---|---|
| 8 mL of water | 4 mL of water | Z31EM588 | Z31EM589 | Z31EM090 |
| 40 μL | 20 μL | 92% | 90% | 37% |
| 80 μL | 40 μL | 88% | 86% | 15% |
| 100 μL | 50 μL | 86% | 83% | 9% |
| 160 μL | 80 μL | 83% | 79% | 2% |
| 200 μL | 100 μL | 81% | 77% | 1% |

| Dilution in | Eq. dilution in | | |
|---|---|---|---|
| 8 mL of water | 4 mL of water | Z31EM433 | Z31EM434 |
| 80 μL | 40 μL | 94% | 93% |

The above results clearly evidence that the compositions according to the invention (Z31EM588 and Z31EM589) lead to high translucence after being diluted in conditions mimicking intravitreal administration. For example, when the emulsions of the invention are diluted in a ratio emulsion/water of 0.01 (80 μL in 8 mL), which corresponds to an intravitreal injection of 40 μL of emulsion, the transmittance is higher than 85%.

Comparative emulsions Z31EM433 and Z31EM434 comprising dexamethasone palmitate solubilized in a triglyceride oil (MCT) also present translucencies that are suitable for intravitreal administration in that they may not cause vision issues such as burring. However, due to their droplet size lower than 100 nm, they do not provide sufficient therapeutic efficacy (cf. Example 3 below).

By contrast, comparative emulsion Z31EM090 does not provide the necessary translucency even when volumes as low as 20 μL (in 4 mL) are administered. Therefore, it is not suitable for intravitreal administration because it would cause troubles of the vision.

### Example 3: Pharmacokinetics (PK) and Pharmacodynamics (PD) Assay

Materials and Methods

The emulsions were prepared as described in Example 1 hereinabove.

The aim of this study was to compare the effects of different dexamethasone palmitate emulsions on VEGF-induced vascular leakage in a rabbit model of blood-retinal barrier (BRB) breakdown over a 6-month study period. Pigmented rabbits from the HY79b strain were randomly divided into groups of six (6) animals (3 males+3 females). Each emulsion was tested on one of the groups. On Day 1, the tested emulsion was administered by intravitreal injection into the right eyes while the left eye remained untreated. At different time points, retinal vascular permeability was expressed as the ratio of the vitreoretinal compartment fluorescence, between the right treated eye and the untreated collateral eye, as measured by ocular fluorimetry 48 h after the right eyes were challenged with 500 ng rhVEGF. Twenty-four (24) weeks after IVT dosing, retina, vitreous and choroid were collected from the right eyes of animals treated with Z31EM588 and Z31EM589 emulsions were collected for bioanalysis assay. Dexamethasone (DXM) and dexamethasone palmitate (DXP) contents were determined in these ocular structures using the RRLC-MS/MS method n° N09F0109.

Results

The pharmacodynamics (PD) data for the 3 doses tested are presented on Table 4. Scale for haze intensity is: ± very very low; + very low; ++: low.

TABLE 4

| | Inj. vol. | Dose | Haze | |
|---|---|---|---|---|
| | (μL) | (μg DXP) | (intensity) | |
| Z31EM588 | 20 | 160 | D1: visible deposit; D2: + | |
| | | | W2: ± 6/6; W4: ± 3/6; W6: 0/6 | |
| | 30 | 240 | D1: visible deposit; D2: ++ | |
| | | | W2: + to ± 6/6; W4: ± 2/6; W5: 0/6 | |
| | 50 | 400 | D7: ++ | |
| | | | W5: + 2/5; W6: - | |
| Z31EM589 | 20 | 160 | D1: visible deposit; D2: + to ++ | |
| | | | W2: ± 3/6; W4: ± 1/6; W5: 0/6 | |
| | 30 | 240 | D1: visible deposit; D2: ++ | |
| | | | W2: + to ± 5/6; W4: + to ± 4/6; | |
| | | | W5: ± 1/6 | |
| | 50 | 400 | D7: ++ | |
| | | | W5: ± 5/6; W6: ± 2/6; W7: - | |
| | | | Normalisation of VEGF-induced | |
| | Inj. vol. | Dose | permeability | |
| | (μL) | (μg DXP) | 3 months | 6 months |
| Z31EM588 | 20 | 160 | 5/6 | 4/5 |
| | 30 | 240 | 6/6 | 5/6 |
| | 50 | 400 | 5/5 | 4/4 |
| Z31EM589 | 20 | 160 | 5/6 | 5/6 |
| | 30 | 240 | 4/4 | 4/4 |
| | 50 | 400 | 5/6 | 5/6 |

The compositions according to the invention (Z31EM588 and Z31EM589) do not induce any significant haze when injected into the vitreous body.

Moreover, intravitreal injection of the compositions according to the invention (Z31EM588 and Z31EM589) lead to efficient treatment by corticosteroid drug dexamethasone at 3-month and 6-month points, as evidenced by the normalization of VEGF-induced permeability (edema resorption). Even at lower doses (20 μL), both emulsions provide effective treatment for at least 6 months.

Following a similar experimental protocol, comparative emulsions Z31EM433 and Z31EM434 (as described in Example 1) has been tested and the results are presented on Table 5.

TABLE 5

| | Inj. | | Normalisation of VEGF-induced | | |
|---|---|---|---|---|---|
| | vol. | Dose | permeability | | |
| | (μL) | (μg DXP) | 1 month | 2 months | 3 months |
| Z31EM433 | 20 | 160 | N.D. | 0/6 | N.D. |
| Z31EM434 | 20 | 160 | N.D. | 0/6 | N.D. |
| | 30 | 240 | 4/6 | 0/6 | N.D. |
| | 40 | 320 | 5/6 | 2/6 | 0/6 |

Although the comparative emulsions Z31EM433 and Z31EM434 comprise the same amount of active ingredient than the emulsions according to the invention (0.8% DXP) and that the amount injected into the vitreous body were similar (160, 240 and 320 μg DXP), the comparative emulsions were not efficient for more than one month. The comparatives emulsions differ from the invention in that they only comprise one surfactant (Cremophor EL®) and thus their droplet size is lower than 100 nm (60 nm).

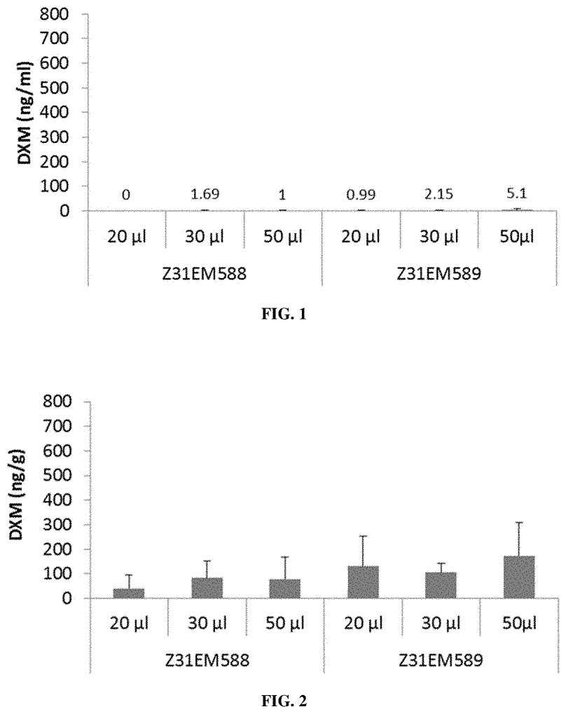

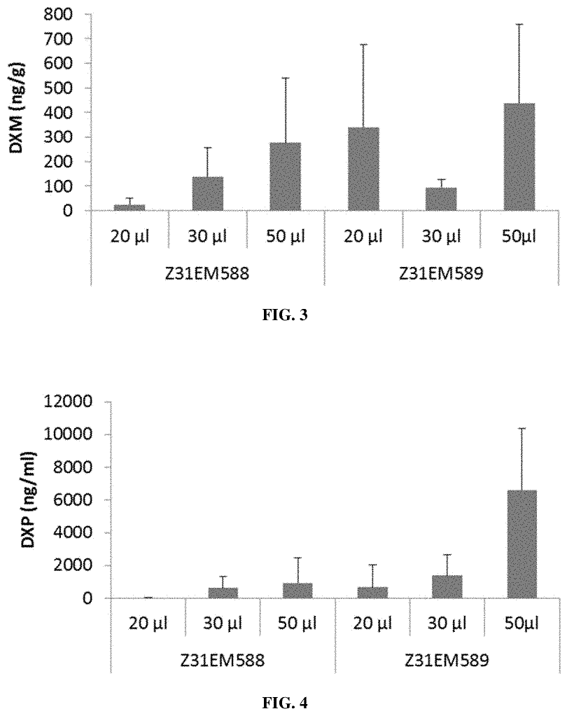

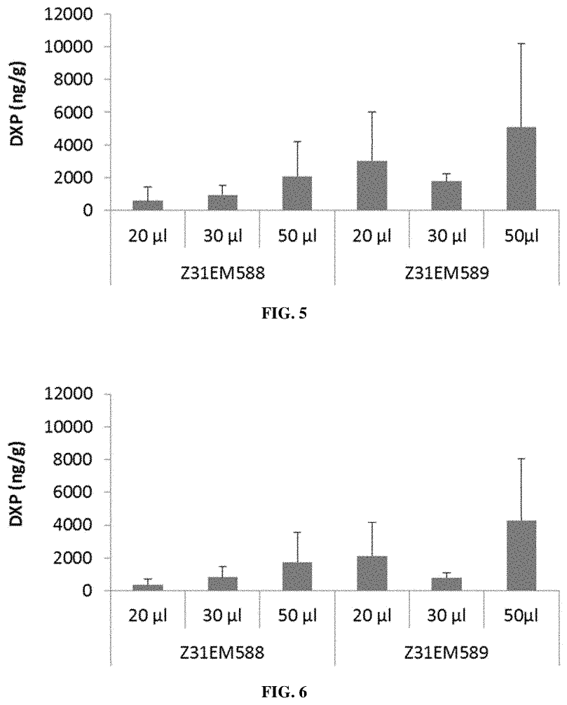

The pharmacokinetics (PK) data for the 3 doses tested of the compositions according to the invention (Z31EM588 and Z31EM589) are presented on FIGS. 1-6. Dexamethasone (DXM) is present in very low concentration in the vitreous body (FIG. 1), whereas it is present in significant amounts in the retina (FIG. 2) and in the choroid (FIG. 3). Therefore, the corticosteroid drug (dexamethasone) is administered to the posterior tissues of the eye (retina, choroid), thereby providing therapeutic activity, without accumulating in the vitreous body where it could induce adverse effects. The corticosteroid prodrug dexamethasone palmitate (DXP) is present in significant amounts in the vitreous body (FIG. 4), in the retina (FIG. 5) and in the choroid (FIG. 6). Therefore, the corticosteroid drug (dexamethasone) is released overtime thanks to the presence of its prodrug (dexamethasone palmitate) in the vitreous body, retina and choroid.

The above results clearly evidence that the compositions according to the invention provide good drug release overtime (controlled and sustained release of the drug) while avoiding troubles of vision and adverse effects. They are thus advantageous for administering active ingredients to the posterior segment of the eye and to treat eye diseases or conditions thereof. It is also evidenced that the droplet size is an essential feature in order to achieve the technical effects of the invention.

### Example 4: Effect of Oil Amount and Surfactants Ratio on the Properties of the Emulsions

Materials and Methods

Materials: The substances used in the preparation of the emulsions were purchased from commercial providers and used without further purification.

Methods—Manufacture of the emulsions: One hundred grams of each emulsions were prepared as described hereinafter: Weighing of the oil phase components in a beaker; Dissolution of the oily phase components under magnetic stirring (200 rpm) and slight heating (50° C.); Heating of the oily phase to 65° C. under magnetic stirring (200 rpm). Parallelly, Weighing of the aqueous phase components in a beaker; Dissolution of the aqueous phase components under magnetic stirring (200 rpm) and slight heating (50° C.); Heating of the aqueous phase to 65° C. under magnetic stirring (200 rpm). Then, Addition of aqueous phase in the oily phase at 65° C. under magnetic stirring (300-400 rpm); Heating of the coarse emulsion to 75° C. under magnetic stirring (300-400 rpm); Dispersion with Polytron homogenizer (Polytron® PT 6100, Kinematica): 5 min at 16000 rpm; Cooling of the emulsion to 25° C. using an ice bath; Continuous homogenization: 10 min 15000 psi with Emulsiflex homogenizer (Avestin® Emulsiflex C₃). The emulsions were then distributed as follows: two 5 mL clear glass vials filled with 5 mL of emulsion for steam sterilisation by autoclave at 121° C. during 20 min (FEDEGARI Autoclavi SPA); the rest of the emulsion was stored in 100 mL clear glass vial. Droplet size: Droplet size was measured using quasi-elastic light scattering after a 1/20 dilution in water (50 μL of emulsion in 950 μL of water) using a High-Performance Particle Sizer being Zetasizer NanoS (Malvern Instruments, UK). Turbidity: The turbidity of each sample (% light transmittance) was measured with a Turbiscan Beckman Coulter (United States) on 8 mL of sample after a 1/100 dilution in water (80 μL of sample in 8 mL of water).

Results

The emulsions with the compositions as shown in Table 6 have been prepared.

TABLE 6

| (% w/w) | Z31EM576 | Z31EM577 | Z31EM578 | Z31EM579 | Z31EM580 |
|---|---|---|---|---|---|
| DXP | 0.80 | 0.80 | 0.80 | 0.80 | 0.80 |
| MCT | 3.00 | 3.00 | 3.00 | 3.00 | 3.00 |
| CrEL | 1.80 | 1.35 | 0.90 | 0.45 | — |
| Span 20 | — | 0.45 | 0.90 | 1.35 | 1.80 |
| Glycerol | 1.00 | 1.00 | 1.00 | 1.00 | 1.00 |
| Water | Qs 100 | Qs 100 | Qs 100 | Qs 100 | Qs 100 |
| (% w/w) | Z31EM566 | Z31EM567 | Z31EM568 | Z31EM569 | Z31EM570 |
| DXP | 1.60 | 1.60 | 1.60 | 1.60 | 1.60 |
| MCT | 6.50 | 6.50 | 6.50 | 6.50 | 6.50 |
| CrEL | 3.90 | 2.93 | 1.95 | 0.98 | — |
| Span 20 | — | 0.98 | 1.95 | 2.93 | 3.90 |
| Glycerol | 1.00 | 1.00 | 1.00 | 1.00 | 1.00 |
| Water | Qs 100 | Qs 100 | Qs 100 | Qs 100 | Qs 100 |
| (% w/w) | Z31EM553 | Z31EM554 | Z31EM555 | Z31EM556 | Z31EM557 |
| DXP | 2.40 | 2.40 | 2.40 | 2.40 | 2.40 |
| MCT | 10.00 | 10.00 | 10.00 | 10.00 | 10.00 |
| CrEL | 6.00 | 4.50 | 3.00 | 1.50 | — |
| Span 20 | — | 1.50 | 3.00 | 4.50 | 6.00 |
| Glycerol | 1.00 | 1.00 | 1.00 | 1.00 | 1.00 |
| Water | Qs 100 | Qs 100 | Qs 100 | Qs 100 | Qs 100 |
| (% w/w) | Z31EM571 | Z31EM572 | Z31EM573 | Z31EM574 | Z31EM575 |
| DXP | 0.80 | 0.80 | 0.80 | 0.80 | 0.80 |
| MCT | 3.00 | 3.00 | 3.00 | 3.00 | 3.00 |
| CrEL | 0.90 | 0.68 | 0.45 | 0.23 | — |
| Tween 20 | — | 0.23 | 0.45 | 0.68 | 0.90 |
| Glycerol | 1.00 | 1.00 | 1.00 | 1.00 | 1.00 |
| Water | Qs 100 | Qs 100 | Qs 100 | Qs 100 | Qs 100 |
| (% w/w) | Z31EM561 | Z31EM562 | Z31EM563 | Z31EM564 | Z31EM565 |
| DXP | 1.60 | 1.60 | 1.60 | 1.60 | 1.60 |
| MCT | 6.50 | 6.50 | 6.50 | 6.50 | 6.50 |
| CrEL | 1.95 | 1.46 | 0.98 | 0.49 | — |
| Tween 20 | — | 0.49 | 0.98 | 1.46 | 1.95 |
| Glycerol | 1.000 | 1.000 | 1.000 | 1.000 | 1.000 |
| Water | Qs 100 | Qs 100 | Qs 100 | Qs 100 | Qs 100 |
| (% w/w) | Z31EM492 | Z31EM550 | Z31EM551 | Z31EM552 | Z31EM546 |
| DXP | 2.40 | 2.40 | 2.40 | 2.40 | 2.40 |
| MCT | 10.00 | 10.00 | 10.00 | 10.00 | 10.00 |
| CrEL | 3.0 | 2.25 | 1.50 | 0.75 | — |
| Tween 20 | — | 0.75 | 1.50 | 2.25 | 3.0 |
| Glycerol | 1.00 | 1.00 | 1.00 | 1.00 | 1.00 |
| Water | Qs 100 | Qs 100 | Qs 100 | Qs 100 | Qs 100 |

DXP: dexamethasone palmitate; MCT: Medium chain triglycerides;
CrEL: Cremophor EL®; Tween 20: Tween® 20; Span 20: Span™ 20.

The aim of this study was to evaluate the effect of the amount of oil phase (oil and active ingredient), ratio of the two surfactants in the mixture and sterilization by autoclave on the properties of the oil-in-water emulsions.

Two different mixtures of non-ionic surfactants were tested: Cremophor EL®/Span™ 20 and Cremophor EL®/Tween® 20. The ratio between the active ingredient (DXP) and the oil (MCT) was maintained constant. The amount of surfactant(s) was increased in relation with the amount of oil so as to obtain stable emulsions.

The properties of the oil-in-water emulsions are presented in Table 7.

TABLE 7

| | | NAC | | AC | |
|---|---|---|---|---|---|
| | Ratio (w/w) | Size [peak] | Turb. | Size [peak] | Turb. |
| (% w/w) | CrEL/Span 20 | (nm) | (%) | (nm) | (%) |
| DXP: 0.8 | 100/0 | 117 | 90 | 146 | 85 |
| | (Z31EM576) | | | | |
| MCT: 3.0 | 75/25 | 141 | 89 | 131 | 89 |
| | (Z31EM577) | | | | |
| Surf.: 1.8 | 50/50 | 156 | 88 | 158 | 86 |
| | (Z31EM578) | | | | |
| | 25/75 | 164 | 84 | 165 | 82 |
| | (Z31EM579) | | | | |
| | 0/100 | 168 | 70 | 180 | 66 |
| | (Z31EM580) | | | | |
| DXP: 1.6 | 100/0 | 119 | 85 | 197 | 18 |
| | (Z31EM566) | | | | |
| MCT: 6.5 | 75/25 | 139 | 85 | 151 | 79 |
| | (Z31EM567) | | | | |
| Surf.: 3.9 | 50/50 | 150 | 84 | 150 | 82 |
| | (Z31EM568) | | | | |
| | 25/75 | 165 | 72 | 178 | 68 |
| | (Z31EM569) | | | | |
| | 0/100 | 166 | 51 | 187 | 46 |
| | (Z31EM570) | | | | |
| DXP: 2.4 | 100/0 | 115 | 79 | PS | PS |
| | (Z31EM553) | | | | |
| MCT: 10.0 | 75/25 | 139 | 77 | 185 | 54 |
| | (Z31EM554) | | | | |
| Surf.: 6.0 | 50/50 | 158 | 74 | 150 | 72 |
| | (Z31EM555) | | | | |
| | 25/75 | 182 | 55 | 171 | 53 |
| | (Z31EM556) | | | | |
| | 0/100 | 208 | 25 | 213 | 22 |
| | (Z31EM557) | | | | |
| DXP: 0.8 | 100/0 | 155 | 80 | 252 | 58 |
| | (Z31EM571) | | | | |
| MCT: 3.0 | 75/25 | 163 | 80 | 158 | 77 |
| | (Z31EM572) | | | | |
| Surf.: 0.9 | 50/50 | 180 | 79 | 166 | 75 |
| | (Z31EM573) | | | | |
| | 25/75 | 174 | 78 | 167 | 77 |
| | (Z31EM574) | | | | |
| | 0/100 | 178 | 78 | 170 | 75 |
| | (Z31EM575) | | | | |
| DXP: 1.6 | 100/0 | 165 | 61 | PS | PS |
| | (Z31EM561) | | | | |
| MCT: 6.5 | 75/25 | 172 | 60 | 208 | 46 |
| | (Z31EM562) | | | | |
| Surf.: 1.95 | 50/50 | 176 | 62 | 172 | 53 |
| | (Z31EM563) | | | | |
| | 25/75 | 181 | 57 | 163 | 54 |
| | (Z31EM564) | | | | |
| | 0/100 | 180 | 55 | 173 | 50 |
| | (Z31EM565) | | | | |
| DXP: 2.4 | 100/0 | 157 | NM | PS | PS |
| | (Z31EM492) | | | | |
| MCT: 10.0 | 75/25 | 173 | 37 | 246 | 15 |
| | (Z31EM550) | | | | |
| Surf.: 3.0 | 50/50 | 200 | 34 | 175 | 29 |
| | (Z31EM551) | | | | |
| | 25/75 | 197 | 45 5 | 173 | 42 |
| | (Z31EM552) | | | | |
| | 0/100 | 205 | 36 | 195 | 30 |
| | (Z31EM546) | | | | |

Surf.: Total surfactants content; Peak: Peak of droplet size distribution; Turb.: Turbidity (% light transmittance); NAC: not autoclaved; AC: sterilization by autoclave; PS: Phase separation (unstable emulsion); NM: Not measured.

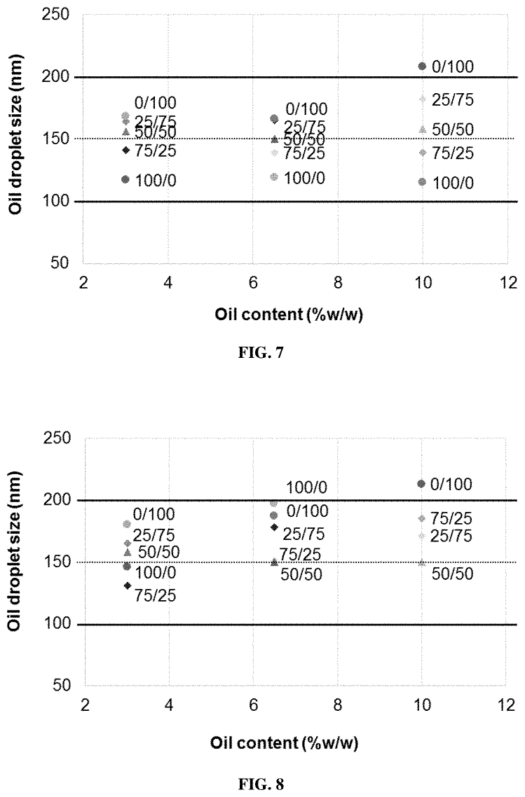

The oil droplet size obtained with the mixtures of CrEL and Span 20 (first part of Table 7 above) are also presented in FIG. 7 (without autoclaving) and FIG. 8 (after autoclaving).

Although the ratio between the surfactants affect the properties of the emulsions, mixtures of two non-ionic surfactants (i.e., ratios in weight ranging from 75/25 to 25/75) lead to droplet sizes ranging from about 100 to about 200 nm, as required in the present invention.

Similarly, using a sorbitan ester (Span 20) or a polysorbate (Tween 20) as cosurfactant of a polyoxyethylene castor oil (CrEL) leads to droplet sizes within adequate working range. The use of a sorbitan ester as cosurfactant is however advantageous because the droplet size is significantly lower and the translucence is typically higher, compared to polysorbate emulsions. The sorbitan ester is especially preferred when high amounts of oil are used (10% w/w).

High oil content (10% w/w) does not prevent the obtention of appropriate droplet size, however lower amounts of oil are advantageous because they lead to a higher translucency and thus further limit the risk of troubles of vision.

By contrast, the emulsions comprising only one non-ionic surfactant generally do not provide satisfying properties in terms of droplet size or translucency, especially after being sterilized.

Consequently, the above results clearly evidence that the technical features of the present invention consistently lead to emulsions meeting the requirements (droplet size, translucency, stability over autoclaving) for intravitreal injection of an active ingredient to the eye, e.g., a steroid prodrug (as described in Example 3 hereinabove).

1-15. (canceled)

16. An oil-in-water emulsion for intravitreal administration comprising:
from about 0.01 to about 50% w/w an oil,
from about 0.001 to about 10% w/w of an active ingredient comprised in said oil,
from about 0.001 to about 25% w/w of a mixture of at least two non-ionic surfactants comprising a polyoxyethylene castor oil, and further comprising a sorbitan ester, a polysorbate, or a mixture thereof, and
water;
wherein said emulsion has a droplet size ranging from about 100 to about 200 nm.

17. The oil-in-water emulsion according to claim 16, wherein said emulsion has a light transmittance after being diluted in water in a ratio in volume emulsion/water of about 0.01 ranging from about 70% to about 100%.

18. The oil-in-water emulsion according to claim 17, wherein said emulsion has a light transmittance after being diluted in water in a ratio in volume emulsion/water of about 0.01 ranging from about 80% to about 100%.

19. The oil-in-water emulsion according to claim 16, wherein said mixture of at least two non-ionic surfactants comprises a polyoxyethylene castor oil and a sorbitan ester.

20. The oil-in-water emulsion according to claim 16, wherein said mixture of at least two non-ionic surfactants comprises polyoxyl-35 castor oil, sorbitan monolaurate, or a mixture thereof.

21. The oil-in-water emulsion according to claim 20, wherein said mixture of at least two non-ionic surfactants comprises polyoxyl-35 castor oil and sorbitan monolaurate.

22. The oil-in-water emulsion according to claim 16, wherein said oil is selected from triglyceride oils.

23. The oil-in-water emulsion according to claim 22, wherein said oil is medium chain triglycerides.

24. The oil-in-water emulsion according to claim 16, wherein said active ingredient is a lipophilic active ingredient.

25. The oil-in-water emulsion according to claim 24, wherein said active ingredient is a long chain ester of a drug.

26. The oil-in-water emulsion according to claim 25, wherein said drug is a corticosteroid.

27. The oil-in-water emulsion according to claim 16, wherein said active ingredient is selected from dexamethasone caprate, dexamethasone laurate, dexamethasone myristate, dexamethasone palmitate, and dexamethasone stearate.

28. The oil-in-water emulsion according to claim 27, wherein said active ingredient is dexamethasone palmitate.

29. The oil-in-water emulsion according to claim 16, wherein said emulsion comprises:
medium chain triglycerides,
an active ingredient selected from dexamethasone caprate, dexamethasone laurate, dexamethasone myristate, dexamethasone palmitate, and dexamethasone stearate, polyoxyl-35 castor oil,
sorbitan monolaurate,
glycerol, and
water.

30. The oil-in-water emulsion according to claim 16, wherein said emulsion is anionic.

31. The oil-in-water emulsion according to claim 16, wherein said emulsion has a droplet size ranging from about 110 to about 175 nm.

32. The oil-in-water emulsion according to claim 31, wherein said emulsion has a droplet size ranging from about 120 nm to about 150 nm.

33. A method for the treatment of an eye disease or condition in a subject in need thereof, wherein said method comprise a step of administering to said subject a therapeutically effective amount of the oil-in-water emulsion according to claim 16.

34. The method according to claim 33, wherein said emulsion is intravitreally injected in an amount ranging from about 5 to about 250 μL.

35. An implantable device or prefilled syringe comprising the oil-in-water emulsion according to claim 16.
